SEQUENCE LISTING (29 sequences)

SEQ ID NO: 1 - DNA, 20 bp, Artificial Sequence
  APOB pcr primer F
gacaacctcaatgctctgct

SEQ ID NO: 2 - DNA, 20 bp, Artificial Sequence
  APOB pcr primer R
tgacttacctggacatggct

SEQ ID NO: 3 - DNA, 30 bp, Artificial Sequence
  APOB SNaPshot primer forward
tttttttttttttgaagaccagccagtgca

SEQ ID NO: 4 - DNA, 21 bp, Artificial Sequence
  NPPA pcr primer f
gccaagagaggggaaccagag

SEQ ID NO: 5 - DNA, 22 bp, Artificial Sequence
  NPPA pcr primer r
agtgagcacagcatcagaaagc

SEQ ID NO: 6 - DNA, 35 bp, Artificial Sequence
  NPPA SNaPshot primer reverse
tttttttttttttttttaatcccatgtacaatgcc

SEQ ID NO: 7 - DNA, 19 bp, Artificial Sequence
  DDAH1 IVS2-33C>T prc primer F
atcctgctttctgcccttt

SEQ ID NO: 8 - DNA, 21 bp, Artificial Sequence
  DDAH1 IVS2-33C>T prc primer r
aagccagtgaagcgtaaacac

SEQ ID NO: 9 - DNA, 40 bp, Artificial Sequence
  DDAH1 IVS2-33C>T SNaPshot primer forward
ttttttttttttttttttttttgtacagtcactggtgcca

SEQ ID NO: 10 - DNA, 22 bp, Artificial Sequence
  FGB -455G>A pcr primer F
aacacacaagtgaacagacaag

SEQ ID NO: 11 - DNA, 20 bp, Artificial Sequence
  FGB -455G>A pcr primer r
gcactcctcaaagagagatg

SEQ ID NO: 12 - DNA, 45 bp, Artificial Sequence
  FGB -455G>A SNaPshot oligo reverse
tttttttttttttttttttttttttttttctatttcaaaaggggc

SEQ ID NO: 13 - DNA, 20 bp, Artificial Sequence
  NPY -52C>G pcr primer f
gttctctctgcgggactggg

SEQ ID NO: 14 - DNA, 20 bp, Artificial Sequence
  NPY -52C>G pcr primer r
ctgccctgggatagagcgaa

SEQ ID NO: 15 - DNA, 50 bp, Artificial Sequence
  NPY -52C>G SNaPshot primer forward
ttttttttttttttttttttttttttttttttgaggagggaggtgctgcg

SEQ ID NO: 16 - DNA, 20 bp, Artificial Sequence
  ADRA2B pcr primer f
gggtgtttgtggggcatctc

SEQ ID NO: 17 - DNA, 19 bp, Artificial Sequence
  ADRA2B pcr primer r
tggcactgcctggggttca

SEQ ID NO: 18 - DNA, 21 bp, Artificial Sequence
  Description of Artificial sequence PCR primer
gagcctgggttcttgggtttc

SEQ ID NO: 19 - DNA, 21 bp, Artificial Sequence
  CBS prc primer r
ggttgtctgctccgtctggtt

SEQ ID NO: 20 - DNA, 25 bp, Artificial Sequence
  snapshot primer cbs forward
ttttttccgcgccctctgcagatca

SEQ ID NO: 21 - DNA, 22 bp, Artificial Sequence
  LPL pcr primer F
cgctccattcatctcttcatcg

SEQ ID NO: 22 - DNA, 22 bp, Artificial Sequence
  LPL pcr primer R
ccccctatcaacagaaacacca

SEQ ID NO: 23 - DNA, 55 bp, Artificial Sequence
  LPL SNaPShot primer
tttttttttttttttttttttttttttttttttttttcttttggctctgacttta

SEQ ID NO: 24 - DNA, 21 bp, Artificial Sequence
  ITGB3 pcr primer F
gcaggaggtagagagtcgcca

SEQ ID NO: 25 - DNA, 21 bp, Artificial Sequence
  ITGB3 pcr primer R
gggcacagttatccttcagca

SEQ ID NO: 26 - DNA, 60 bp, Artificial Sequence
  ITGB3 SNaPshot primer reverse
tttttttttttttttttttttttttttttttttttttttttgtcacagcgaggtgagccc

SEQ ID NO: 27 - DNA, 22 bp, Artificial Sequence
  NPPA pcr primer F
ttagcagttcatattcctcccc

SEQ ID NO: 28 - DNA, 20 bp, Artificial Sequence
  NPPA pcr primer R
agcctcttgcagtctgtccc

SEQ ID NO: 29 - DNA, 65 bp, Artificial Sequence
  NPPA SNaPshot primer reverse
ttttttttttttttttttttttttttttttttttttttttttttttctccctggctgttatcttc

(19) United States
(12) Patent Application Publication
(10) Pub. No.: US 2006/0166205 A1
(43) Pub. Date: Jul. 27, 2006

(54) Detecting the risk of cardiovascular disease by detecting mutations in genes, including genes encoding a2b-adrenoceptor and apoliporotein b

(75) Inventors: Jukka T Salonen, Kuopio (FI); Tomi-Pekka Tuomainen, Kuopio (FI); Mia Pirskanen, Kuopio (FI)

Correspondence Address: BIRCH STEWART KOLASCH & BIRCH, PO BOX 747, FALLS CHURCH, VA, 22040-0747, US

(73) Assignee: OY JURILAB LTD, Kuopio (FI)

(21) Appl. No.: 10/530,347

(22) PCT Filed: Oct. 7, 2003
(86) PCT No.: PCT/FI03/00740

§ 371 Date: Mar. 3, 2006

(30) Foreign Application Priority Data
Oct. 7, 2002 (FI) 20021783

(51) Int. Cl. C12Q 1/68; C12P 19/34

(52) U.S. Cl. 435/6; 435/91.2

(57) ABSTRACT

The present invention provides a method of identifying subject's susceptibility to cardiovascular diseases or risk of developing myocardial infarction (MI) or cerebrovascular stroke by detecting gene polymorphisms and other gene mutations from a biological sample of the subject and optionally obtaining information concerning the family and medical history, blood, serum, plasma and urinary analytes of the subject. The invention also provides a multivariate model, a combination or algorithm of variables which best describes the probability of cardiovascular diseases, especially MI and stroke. The invention also relates to a test kit and software for accomplishing the method.

The present invention provides a method of identifying subject's susceptibility to cardiovascular diseases or risk of developing myocardial infarction (MI) or cerebrovascular stroke by detecting gene polymorphisms and other gene mutations from a biological sample of the subject and optionally obtaining information concerning the family and medical history, blood, serum, plasma, urinary analytes and clinical findings of the subject. The invention also provides a multivariate model, a combination or algorithm of variables which best describes the probability of cardiovascular diseases, especially MI and stroke. The invention also relates to a test kit and software for accomplishing the method.

### FIELD OF THE INVENTION

The present invention is generally directed to a method for assessing the risk of myocardial infarction (MI) and cerebrovascular stroke in an individual, such as a human. Specifically, the invention is directed to a method that utilises both genetic and phenotypic information as well as information obtained by questionnaires to construct a score that provides the probability of developing an MI or stroke. Furthermore, the invention provides a kit for carrying out the method. The kit can be used to set an etiology-based diagnosis of cardiovascular diseases for targeting of treatment and preventive interventions, such as dietary advice as well as stratification of the subject in clinical trials testing drugs and other interventions.

### BACKGROUND OF THE INVENTION

The coronary heart disease (CHD) and cerebrovascular disease are multifactorial diseases and the leading causes of morbidity, death and disability globally. Even though the age-standardized incidence of and mortality from CHD and stroke are still declining in the Western world, the number of cardiovascular events and subsequent hospitalizations and expenditure are increasing, due to the elevation of life expectancy of the population. It has been estimated based on twin and migration studies that the heritability of CHD and stroke is of the order of 50-60% and there are no major gene effects. Thus, multiple genes and non-genetic risk factors contribute to the development and progression of CHD. Different clinical manifestations of CHD (i.e. angina pectoris, myocardial infarction, sudden death) and stroke have overlapping but also somewhat distinct pathophysiology and risk factors.

CHD and stroke may be caused in different individuals by different reasons and through different pathophysiologic pathways. Often, however, the same risk factors and pathways are operating, but their importance for each individual varies. Regarding pathophysiology, CHD and stroke may be caused by obstruction of the coronary (cerebrovascular) arteries, vasoconstriction or vasospasm in these, thrombotic phenomena or arrhythmias. Coronary and cerebrovascular arterial obstruction is most often caused by atheroma formation. This is a complex disease, but lipids and their metabolism such as oxidation plays a key role. Other major factors leading to atheroma formation are tobacco smoking, hypertension, diabetes, obesity and hyperhomocysteinemia. Additional risk factors include elevated coagulation factors, platelet activation and decreased nitric oxid availability. Men, older persons and those with a family history of CHD are at elevated risk.

Persons who have mutations in genes regulating lipids, their metabolism, blood pressure, platelet functions, coagulation, fibrinolysis, homocysteine metabolism and the function of the cardiac muscle can be expected to be at an elevated risk of CHD. Assessing a number of these mutations can be used to predict MI and cerebrovascular stroke.

A number of meta-analyses have studied multivariate risk functions from diverse populations in the prediction of CHD. None of these have concerned the effects of specific genotyped gene mutations. A recent meta-analysis concerned ordering risk, magnitude of relative risks, and estimation of absolute risk in prospective cohort studies (Diverse Populations Collaborative Group 2002). The outcome measure was death from CHD. The analysis included 105 420 men and 56 535 women 35-74 years of age and free of CHD at baseline from 16 observational studies with a total of 27 analytical groups. The area under the receiver operating characteristic curve (AUC) was used to judge the ability of the multivariate risk function to order risk correctly. The AUCs differed significantly between the studies (p<0.01) but were very similar for different risk functions applied to the same population, indicating similar ability to rank risk for different models. The magnitudes of the relative risks associated with major risk factors (age, systolic blood pressure, serum total cholesterol, smoking, and diabetes) varied significantly across studies (p<0.05 for homogeneity). The prediction of absolute risk was not very accurate in most of the cases when a model derived from one study was applied to a different study. The authors concluded that when considered qualitatively, the major risk factors are associated with CHD mortality in a diverse set of populations.

The new Sheffield table and modified joint British societies coronary risk prediction (JBS) chart are widely used (Rabindranath et al. 2002). The JBS chart approximates age and systolic blood pressure, and the new Sheffield table dichotomises blood pressure, and these simplifications may lead to diagnostic inaccuracy. Methods: The diagnostic performance of the charts against an individualised laboratory based CHD risk calculation in 1102 subjects in primary care were evaluated and compared. The new Sheffield table and modified JBS chart performed equally well.

Most previously used models used to predict individual risk of death from coronary heart disease (CHD) were developed from data of three decades ago from the Framingham Heart Study. CHD mortality rates have declined markedly since that period as a result of improvement in both risk factor status and medical interventions. Generalization of the results from this one study to the population at large remains a matter of concern. Liao and coworkers (1999) compared predictive functions derived from the major risk factors for CHD from Framingham and two more recent American cohorts, the First and Second National Health and Nutrition Examination Survey (NHANES I and NHANES II). The participants included 1846 men and 2323 women 35 to 69 years of age and free of CHD at the fourth examination (1954 to 1958) from the Framingham Study; 2753 men and 3858 women from the NHANES I (1971 to 1975); and 2655 men and 3050 women from NHANES II (1976 to 1980). The three cohorts were monitored for 24, 20, and 15 years, respectively. Significant heterogeneity existed among studies in the magnitude of the Cox coefficients for the individual factors (ie, age, systolic blood pressure, serum total cholesterol, and smoking status), especially among men. When risk factors were considered collectively, however, functions derived from and applied to different cohorts had a similar ability to rank individual risk. The areas under the receiver operating characteristic curves were 0.71 to 0.76 in men and 0.76 to 0.81 in women when different risk functions were applied to their own population or to a second population. The cumulative CHD survival observed in women in the two cohorts was close to what was predicted from the Framingham equation. The authors concluded that the Framingham risk model for the prediction of CHD mortality rates provides a reasonable rank ordering of risk for individuals in the US white population for the period 1975 to 1990. However, prediction of absolute risk is less accurate.

### SUMMARY OF THE INVENTION

The object of the present invention is a method of identifying the risk of cardiovascular diseases, especially MI and stroke, by detecting gene polymorphisms and other gene mutations from a biological sample of the subject. The information obtained from this method can be combined with other information concerning an individual, e.g. results from blood measurements, clinical examination and questionnaires. The genetic information includes data on mutations in genes associated with MI and/or stroke. The blood measurements include the determination of blood or plasma or serum analytes that predict CHD or stroke such as blood lipid, homocysteine, glucose, and insulin concentrations and urinary excretion of nicotine metabolites. The information to be collected by questionnaire includes information concerning gender, age, family and medical history and health-related habits such as smoking. Clinical information collected by examination includes e.g. information concerning height, weight, hip anf waist circumference, systolic and diastolic blood pressure, heart rate, other electro-/audiographic parameters and maximal oxugen uptake.

The invention particularly provides a method for diagnosing a susceptibility to cardiovascular disease especially myocardial infarction (MI) and stroke in a subject by detecting genetic variation or polymorphism, i.e. a mutation, in at least three of the genes selected from the group consisting of:
(a) α_{2B}-adrenoceptor
(b) apolipoprotein B
(c) dimethylarginine dimethylaminohydrolase 1
(d) fibrinogen-beta
(e) neuropeptide Y
(f) natriuretic peptide precursor A
(g) cystathione beta synthase
(h) glycoprotein IIb/IIIa
(i) lipoprotein lipase
comprising the steps of:
   i) providing a biological sample of the subject to be tested,
   ii) detecting the presence of mutations in the genes, the presence of a mutation in one or several of the genes indicating an increased risk of coronary heart disease (CHD) and/or myocardial infarction (MI) in said subject.

### DETAILED DESCRIPTION OF THE PREFERRED EMBODIMENTS OF THE INVENTION

In a preferred embodiment the invention comprises the combination of information from a large number of variables (measurements) to predict the probability of MI and stroke. The predictor information includes an assessment of genotypes and haplotypes in genomic DNA and optionally data obtainable by interviews, questionnaires, clinical examination and/or blood analyte measurements. This predictor information can be collected in any age. This method is also applicable to middle-aged persons.

Information concerning genomic DNA genotypes concerns polymorphisms such as single nucleotide polymorphisms (SNPs) and mutations in e.g. the following genes (OMIM abbreviations): APOA1, APOA2, APOA4, APOB, APOC1, APOC2, APOC3, APOC4, APOD, APOE, ARG, LDLR, OLR1, MSR1, MSR2, LPA, LPL, LIPC, LIPG, CETP, ETL, GPIIIa, ICAM1, ICAM2, ICAM3, SELL, SELE, MMP1, MMP3, ITGB n, ADD1, ADD2, ADD3, NPY, NPY1R, NPY2R, NPY3R, NPY4R, NPY5R, HFE1, HFE2, HFE3, TFRC, TFR2, PON1, PON2, SOD1, SOD2, SOD3, CAT, GSTM1, GSTM2, GSTM3, GSTP1, GPX1, GPX3, TNF, TNFB, TRX, NOS3, NOS3, DDAH1, DDAH2, ADRB1, ADRB2, ADRB3, F2, F5, F7, F8, F13, VWF, PAI1, PAI2, FGA, FGB, FGG, ACE, AGT, AGTR1, ATG, SCAP, SCNN1A, SCNN1B, NPPA, CBS, MTHFR, or any other candidate genes that will be observed to relate to the susceptibility to MI or stroke.

The data that can be obtained by questionnaire, interview or clinical examination includes information concerning:
1) age,
2) gender,
3) medical history, i.e. prevalent diseases,
4) family history, i.e. diseases of parents and siblings,
5) tobacco smoking,
6) alcohol use,
7) physical activity and exercise,
8) high weight or obesity in childhood and adolescence,
9) personality traits such as depression, anxiety, hostility,
10) psychological and mood states such as anger, irritability,
11) low self-esteem or weak self-image,
12) lack of social skills, social isolation, lack of social networks,
13) self-image promoting alcohol use (e.g. easy-taking),
14) adulthood socioeconomic circumstances (e.g. being single, divorced or widowed as the marital status, posessing no phone, low socioeconomic status, unemployment and urban place of residence,
15) stressful life events,
16) coping styles, coping capacity, anger control,
17) history of diabetes,
18) high perceived cardiovascular risk,
19) high amount of hospitalizations, poor health status,
20) blood pressure, heart rate, maximal oxygen uptake,
21) other relevant information that can be collected by self-administered questionnaire, by an interview or by clinical examination of the subject.

Information obtainable by measurements from blood, blood cell, plasma, serum or urine samples includes:
1) serum or plasma cholesterol, HDL and LDL cholesterol,
2) serum or plasma triglycerides,
3) serum or plasma apolipoproteins,
4) serum or plasma insulin concentration,
5) blood or serum glucose concentration,
6) blood hemoglobin concentration,
7) serum ferritin or transferring receptor concentrations,
8) serum fibrinogen and other coagulation factor concentration,
9) measurement of platelet activation, aggregation and/or adhesion,
10) serum or plasma concentrations of inflammatory markers such as CRP,
11) other relevant information that can be obtained by chemical or biochamical measurements.

Numerous genotyping methods have been described in the art for analysing nucleic acids for the presence of specific sequence variations e.g. SNP's, insertions and deletions (for review see Syvanen 2001 and Nedelcheva Kristensen et al. 2001). In these methods a sample containing nucleic acid (e.g. blood, tissue biopsy or buccal cells) is obtained from the patient and the sequence variations of interest are identified and visualised from the nucleic acids.

Allelic variants in genes can be discriminated by enzymatic methods (with the aid of restriction endonucleases, DNA polymerases, ligases etc.), by electrophoretic methods (e.g. single strand conformation polymorphism (SSCP), heteroduplex analysis, fragment analysis and DNA sequencing), by solid-phase assays (dot blots, microarrays, microparticles, microtiter plates etc.) and by physical methods (e.g. hybridisation analysis, mass spectrometry and denaturing high performance liquid chromatography (DHPLC)). In most of the genotyping assays different polymerase chain reaction (PCR) applications are used both to increase the signal to noise ratio as well as spare sample nucleic acid before allele discrimination. Detectable labels (fluorochromes, radioactive labels, biotin, modified nucleotides, haptens etc) can be used to enhance visualization of allelic variants.

This invention is based on the principle that a small number of genotypings are performed, and the mutations to be typed are selected on the basis of their ability to predict MI and/or stroke. For this reason any method to genotype mutations in a genomic DNA sample can be used. If non-parallel methods such as real-time PCR are used, the typings are done in a row. The PCR reactions may be multiplexed or carried out separately in a row or in parallel aliquots.

The score that predicts the probability of MI or stroke may be calculated using a multivariate failure time model or a logistic regression equation as follows: Probability of a cardiovascular disease=[1+e^{(-(-a+Σ(bi*xi))}]⁻¹, wherein e is Napier's constant, Xᵢ are variables related to the cardiovascular disease, bᵢ are coefficients of these variables in the logistic function, and a is the constant term in the logistic function. The model may additionally include any interaction (product) or terms of any variables Xᵢ, e.g. bᵢXᵢ. An algorithm is developed for combining the information to yield a simple prediction of MI as percentage of risk in 10 years. An alternative statistical model is a failure-time model such as the Cox's proportional hazards' model.

Experimental Section

Determining Individual Genotypes with SNaPShot

The method according to the invention for the determination of the allelic pattern of the codons/mutations in question can be carried out with polymerase chain reaction (PCR) in combination with, for example, an allele specific primer extension method (SNaPshot, Applied Biosystems) or DNA fragment analysis followed by capillary electrophoresis with ABI Prism 3100 Genetic Analyzer (Applied Biosystems).

In a SNaPshot reaction the genomic DNA region containing the mutation is question is amplified with PCR. The amplified PCR reaction is purified and the product is used as a template in SNaPshot reaction.

For the SNaPshot reaction an extension primer that ends one nucleotide 5′ of a given single nucleotide polymorphism (SNP) locus is designed. In the SNaPshot reaction the extension primer binds to its complementary template in the presence of fluorescent labelled dideoxy-NTPs ([F]ddNTPs) and DNA polymerase. The polymerase extends the primer by only one nucleotide, adding a single [F]ddNTP to its 3′ end. In the analysed data nucleotide A is seen in green colour, C is seen in black colour, G is seen in blue colour and T in red colour. If for example the genotype is A/A then only green colour is detected. For a heterozygous A/C green and black colour are detected.

When multiple SNPs are determined in the same reaction, the extension primers need to differ significantly in length (4-6 nucleotides) to avoid overlap between the final SNaPshot products. This can be accomplished by adding a variable number of nucleotides dT, dA, dC or cGATC to the 5′ end of the different extension primers. The different SNPs can then be detected in the capillary electrophoresis according to the different size of the SNaPshot product. To perform SnaPshot genotyping under standard conditions, refer to the user manual (ABI Prism SnaPshot Multiplex kit, Protocol, Applied Biosystems).

In the DNA fragment analysis, a fluorescent lable is attached to the 5′ end of the PCR primer. In the DNA fragment analysis, the alleles of the locus to be genotyped are different in length (i.e. there is a deletion or an insertion of known number of nucleotides in the studied locus). The different alleles can then be detected after the capillary electrophoresis due to the different migration rates of the different lengths of the pcr product (i.e. alleles).

Polymerase Chain Reaction (PCR)

The genomic DNA regions containing the mutations in question can be amplified with PCR either in separate reactions or all in one single reaction mix (i.e. multiplex PCR) with PTC-220 DNA Engine Dyad PCR machine (MJ Research). The PCR amplification was conducted in a 20 μl volume: the reaction mixture contained 60 ng human genomic DNA nucleotides (dATP, dCTP, dGTP, dTTP), 0.5 μM of each primers and 1 unit of the DNA polymerase (QIAGEN, Hot Start Taq DNA polymerase). The PCR conditions need to be determined experimentally, and the following standard protocol can be used as a start: first the reaction was hold 10 minutes at 94° C., then the following three steps were repeated for 35 cycles: 45 seconds at 94° C., 45 seconds at 55° C., 1 minute 30 seconds at 72° C., after which the reaction was kept at 72° C. for an additional 5 minutes and finally hold at 4° C.

APOB Thr98Ile (Also Known as APOB Thr71Ile)

The nucleotide sequence of the primer pair for the amplification of human APOB gene (apolipoprotein B gene) Thr98Ile mutation (SEQ ID NO:1) (SEQ ID NO:3) (also known as Thr71Ile mutation) was as follow: 5′-GAC AAC CTC AAT GCT CTG CT-3′ (SEQ ID NO:5) and 5′-TGA CTT ACC TGG ACA TGG CT-3′ (SEQ ID NO:6).

NPPA Val32Met

The nucleotide sequence of the primer pair for the amplification of human NPPA (natriuretic peptide precursor A gene) (gene is also known as ANF or ANP or PND or Pronatriodilatin (atrial natriuretic peptide)) Val32Met mutation (SEQ ID NO:8) (SEQ ID NO:10) was as follow: 5′-GCC AAG AGA GGG GAA CCA GAG-3′ (SEQ ID NO:X12) and 5′-AGT GAG CAC AGC ATC AGA AAG C-3′ (SEQ ID NO:13).

DDAH1 IVS2-33C>T

The nucleotide sequence of the primer pair for the amplification of human DDAH1 (dimethylarginine dimethylaminohydrolase 1) IVS2-33C>T mutation (in the following sequences SEQ ID NO:15 and SEQ ID NO:16 the IVS2-33C>T polymorphism is located at the position 1041) was as follows: 5′-ATC CTG CTT TCT GCC CTT T-3′ (SEQ ID NO:17) and 5′-AAG CCA GTG AAG CGT AAA CAC-3′ (SEQ ID NO:18).

FGB-455G>A

The nucleotide sequence of the primer pair for the amplification of human FGB gene (fibrinogen-beta gene) promoter mutation-455G>A mutation (In the following sequences SEQ ID NO:20 and 21 the FGB-455G>A polymorphism is located at the position 1437) (SEQ ID NO:20) (SEQ ID NO:21) was as follow: 5′-AAC ACA CAA GTG AAC AGA CAA G-3′ (SEQ ID NO:22) and 5′-GCA CTC CTC AAA GAG AGA TG-3′ (SEQ ID NO:23).

NPY-52C>G

The nucleotide sequence of the primer pair for the amplification of human NPY gene (neuropeptide Y gene)-52 C>G mutation (in the following sequences SEQ ID NO:25 and 26 the NPY-52C>G polymorphism is located at the position 1000) (SEQ ID NO:25) (SEQ ID NO:26) was as follow: 5′-GTT CTC TCT GCG GGA CTG GG-3′ and (SEQ ID NO:27) 5′-CTG CCC TGG GAT AGA GCG AA-3′ (SEQ ID NO:28).

CBS Ile278Thr

The nucleotide sequence of the primer pair for the amplification of human CBS gene (cystathionine-beta-synthase gene) Ile278Thr mutation (SEQ ID NO:36, SEQ ID NO:38) was as follow: 5′-GAG CCT GGG TTC TTG GGT TTC-3′ (SEQ ID NO:40) and 5′-GGT TGT CTG CTC CGT CTG GTT-3′ (SEQ ID NO:41).

LPL Asn318Ser (Also Known as LPL Asn291 Ser mutation)

The nucleotide sequence of the primer pair for the amplification of human LPL gene (lipoprotein lipase gene) Asn318Ser mutation (SEQ ID NO:43) (SEQ ID NO:45) (also known as LPL Asn291Ser mutation) was as follow: 5′-CGC TCC ATT CAT CTC TTC ATC G-3′ (SEQ ID NO:47) and 5′-CCC CCT ATC AAC AGA AAC ACC A-3′ (SEQ ID NO:48).

ITGB3 Leu59Pro (Also Known as Leu33Pro Mutation)

The nucleotide sequence of the primer pair for the amplification of human ITGB3 (integrin, beta 3, (platelet glycoprotein IIIa, antigen CD61) Leu59Pro mutation (SEQ ID NO:50) (SEQ ID NO:52) (also known as Leu33Pro mutation) was as follow: 5′-GCA GGA GGT AGA GAG TCG CCA-3′ (SEQ ID NO:54) and 5′-GGG CAC AGT TAT CCT TCA GCA-3′ (SEQ ID NO:55).

NPPA OPA152Arg

The nucleotide sequence of the primer pair for the amplification of human NPPA (natriuretic peptide precursor A gene) (gene is also known as ANF or ANP or PND or Pronatriodilatin (atrial natriuretic peptide)) OPA152Arg mutation (SEQ ID NO:57) (SEQ ID NO:59) was as follow: 5′-TTA GCA GTT CAT ATT CCT CCC C-3′ (SEQ ID NO:61) and 5′-AGC CTC TTG CAG TCT GTC CC-3′ (SEQ ID NO:62).

Purification of the PCR Products for SNaPshot Reaction

The PCR products were purified with SAP (Shrimp Alkalinen Phosphatase, USB Corporation) and ExoI (Exonuclease I, USB Corporation) treatment. This was done to avoid the participation of the unincorporated dNTPs and primers from the PCR reaction to the subsequent primer-extension reaction. More specifically 5 units of SAP and 2 units of Exol were added to 15 μl of the PCR product. Reaction was mixed and incubated at 37° C. for 1 hour. After that the reaction was incubated at 75° C. for 15 minutes to inactivate the enzymes and afterwards kept at 4° C.

Primer Extension Reaction (SNaPshot Reaction)

In the subsequent primer extension reaction (SNaPshot reaction) 5 μl of SNaPshot Multiplex Ready Reaction Mix (Applied Biosystems), 3 μl of purified PCR products, 1 μl of pooled extension primers (depending of the signal in the SNaPshot reaction, the primer concentrations in the mix can range between 0.05 μM and 1 μM) and 1 μl water are mixed in a tube. The reaction is incubated at 94° C. for 2 minutes and then subject to 25 cycles of 95° C. for 5 s, 50° C. for 5 s and 60° C. for 5 s in a PTC-220 DNA Engine Dyad PCR machine (MJ Research).

The nucleotide sequence of the extension primer for the genotyping of human APOB Thr71Ile mutation in a SNaPShot reaction was as follow: 5′-TTT TTT TTT TTT TGA AGA CCA GCC AGT GCA-3′ (SEQ ID NO:7).

The nucleotide sequence of the extension primer for the genotyping of human NPPA Val32Met mutation in a SNaPShot reaction was as follow: 5′-TT TTT TTT TTT TTT TTT AAT CCC ATG TAC AAT GCC-3′ (SEQ ID NO:14).

The nucleotide sequence of the extension primer for the genotyping of the human DDAH1 IVS2-33C>T mutation in a SNaPShot reaction was as follow: 5′-T TTT TTT TTT TTT TTT TTT TTT GTA CAG TCA CTG GTG CCA-3′ (SEQ ID NO:19).

The nucleotide sequence of the extension primer for the genotyping of human FGB promoter-455G>A mutation in a SNaPshot reaction was as follow: 5′-T TTT TTT TTT TTT TTT TTT TTT TTT TTC TAT TTC AAA AGG GGC-3′ (SEQ ID NO:24).

The nucleotide sequence of the extension primer for the genotyping of human NPY gene-52 C>G mutation in a SNaPShot reaction was as follow: 5′-T TTT TTT TTT TTT TTT TTT TTT TTT TTT TTT GAG GAG GGA AGG TGC TGC G-3′ (SEQ ID NO:29).

The nucleotide sequence of the extension primer for the genotyping of human LPL Asn291Ser mutation was as follow: 5′-TTT TTT TTT TTT TTT TTT TTT TTT TTT TTT TTT TTT TCT TTT GGC TCT GAC TTT A-3′ (SEQ ID NO:49)

The nucleotide sequence of the extension primer for the genotyping of human ITGB3 Leu33Pro mutation was as follow: 5′-TT TTT TTT TTT TTT TTT TTT TTT TTT TTT TTT TTT TTT TTT GTC ACA GCG AGG TGA GCC C-3′ (SEQ ID NO:56).

The nucleotide sequence of the extension primer for the genotyping of human NPPA OPA152Arg mutation was as follow: 5′-T TTT TTT TTT TTT TTT TTT TTT TTT TTT TTT TTT TTT TTT TTT TTT CTC CCT GGC TGT TAT CTT C-3′ (SEQ ID NO:63).

Post-Extension Treatment

After the primer extension reaction 1 unit of SAP was added to the reaction mix and the reaction was incubated at 37° C. for 1 hour. The enzyme was inactivated by incubating the reaction mix at 75° C. for 15 minutes. Afterwards the samples were placed at 4° C. The post-extension treatment was done to prevent the unincorporated fluorescent ddNTPs obscuring the primer extension products (SNaPshot products) during electrophoresis with ABI Prism 3100 Genetic Analyzer.

DNA Fragment Analysis of ADRA2B Insertion/Deletion Polymorphism

ADRA2B Insertion/Deletion Mutation

ADRA2B gene (alpha2B-adrenergic receptor gene) insertion/deletion polymorphism (SEQ ID NO:30) (SEQ ID NO:32) was as follows 5′-GGG TGT TTG TGG GGC ATC TC-3′ (SEQ ID NO:34) and 5′-TGG CAC TGC CTG GGG TTC A-3′ (SEQ ID NO:35). A fluorescent label has been added to the 5′ end of one of the above mentioned pcr primers. Thus, the pcr fragment is detectable in the capillary electrophoresis conducted with ABI Prism 3100 Genetic Analyzer.

The insertion/deletion polymorphism of ADRA2B gene concerns an insertion or an deletion of three glutamic acids in the region of 12 Glu aminoacids in the codons 298-309 (SEQ ID NO:30). Thus depending on the genotype, there is either 9 Glu (deletion) or 12 Glu (insertion) at the ADRA2B locus. Depending on whether the amplified allele had an insertion or a deletion in the studied locus, the size of the pcr product was 91 bp (insertion allele) or 82 bp (deletion allele). Thus, for homotzygotes (insertion/insertion or deletion/deletion) only one size of a fragment was detected either 91 bp or 82 bp, respectively. For heterotzygotes both of the above mentioned fragments were detected.

Capillary Electrophoresis with ABI Prism 3100 Genetic Analyzer

Aliquots of 1 μl of pooled SNaPshot products, 0.5-1.0 μl of the ADRA2B insertion/deletion pcr product, 9.00 μl of Hi-Di formamide (Applied Biosystems) and 0.25 μl GeneScan-120 LIZ size standard (Applied Biosystems) were combined in a 96-well 3100 optical microamp plate (Applied Biosystems). The reactions were denatured by placing them at 95° C. for 5 minutes and then loaded onto a ABI Prism 3100 Genetic Analyzer (Applied Biosystems). Elelctrophoresis data was processed and the genotypes were visualized by using the GeneScan Analysis version 3.7 (Applied Biosystems).

Testing the Risk of MI and Stroke

Risk factors for MI and stroke were studied in the KIHD cohort. Briefly, the “Kuopio Ischaemic Heart Disease Risk Factor Study” (KIHD) is a prospective population study in men in Eastern Finland (Salonen 1988, Tuomainen et al. 1999). The study protocol for KIHD was approved by the Research Ethics Committee of the University of Kuopio. The study sample comprised men from Eastern Finland aged 42, 48, 54 or 60 years. A total of 2682 men were examined during 1984-89. All participants gave a written informed consent. The follow-up of coronary and cerebrovascular events was to the end of 2000, providing an average follow-up time of 13.4 years. Genotypings were carried out for approximately 1600 men, resulting to over 21,000 person-years of follow-up.

Of the baseline examination participants, 1038 men were re-examined approximately four years after the baseline survey, in 1991-3. The mean follow-up time was 4.1 years. Of 1177 eligible men, 139 could not be contacted or refused to participate, 1038 (88.2%) men participated.

A nested case-control set was selected consisting of 47 men who developed a MI by the end of 2000 and 47 control men matched for age, place of residence, fasting time and examination day, who had no MI by the end of 2000. Both the cases and the controls had no MI prior to the 1991-3 examination. Similarly, a case-control set of 22 men who had a stroke during the follow-up and 22 identically matched controls were selected. Neither group had a previous stroke prior to the 1991-3 examination. A large number of genotypings were carried out in these nested case-control sets.

Data on CHD and cerebrovascular disease during the follow-up were obtained by computer linkage to the national computerized hospital discharge registry. Diagnostic information was collected from the hospitals and all heart attacks and cerebrovascular events were classified according to rigid predefined criteria. The diagnostic classification of acute coronary events was based on symptoms, electrocardiographic findings, cardiac enzyme elevations, autopsy findings and the history of CHD. Each suspected coronary event (ICD-9 codes 410-414 and ICD-10 codes I20-I25) was classified into 1) a definite acute myocardial infarction (AMI), 2) a probable AMI, 3) a typical acute chest pain episode of more than 20 minutes indicating CHD, 4) an ischemic cardiac arrest with successful resuscitation, 5) no acute coronary event or 6) an unclassifiable fatal case. The categories 1) to 3) were combined for the present analysis to denote MI. Cerebrovascular events were classified according to the FINNMONICA criteria.

The purpose of this project was to develop a simple gene test that can be used to diagnose CHD and cerebrovascular disease and to predict the risk of acute myocardial infarction and stroke in healthy and sick persons. We had several data sets available to us for this work. The model was constructed in a prospective nested case-control set of 50 men who did not have prior MI but developed an MI during a 8-year follow-up, and 50 age-matched control men who did not develop MI during the follow-up. This case-control set was derived from the KIHD 1991-3 examination, in which over 1000 men aged 46-64 from Eastern Finland years were examined (see ref. 4). We typed over 100 mutations assumed to be relevant regarding CHD and stroke in DNA samples obtained at baseline, and collected phenotypic information yielding over 5000 variables.

Of the about 100 mutations, the four most predictive ones of MI were selected using hierarchial step-up binary logistic modelling (Table 1). These predicted 61% of future MIs (R square 16%). Theoretically (based on twin studies), this is the maximal prediction that can be achieved by genes. The second step was to find the most predictive other variables. We tested similarly over 1000 variables including all known risk factors for CHD. A set of six variables (Table 1) was defined that increased the prediction to 80% (R square 53%), and the predicted probability of MI for each person varied from 0.0002 to 0.9991. These can be recorded using five simple questions and measuring waist and hip circumferences. None of the over 200 biochemical measurements tested contributed much additional information to the model. The same concerned blood pressure and other clinical measurements. Age and gender are additionally needed in the model.

We also constructed a 3-gene model which with four questionnaire variables predicted 80% (R square 55%) of cerebrovascular strokes (Table 2).

Thus, we invented a 10-variable model that predicted future myocardial infarction and a 7-variable model that predicted stroke very well in the data set they were derived of. The prediction of 80% is higher than in any published epidemiologic cohort study. An advantage is that only a small number of genotypings need to be carried out and a very short self-administered questionnaire needs to be filled in. One of the mutations in both tests is the same, so in total only six genotypings are needed to predict both MI and stroke.

TABLE 1
A multivariate logistic model predicting the risk of MI.

| | | Coefficient | | | |
|---|---|---|---|---|---|
| Predictor | Mutation | (bᵢ) | S.E. | p-value | Odds ratio |
| Natriuretic peptide precursor A (met carrier | Val32Met | 3.133 | 1.713 | 0.068 | 22.9 |
| vs. non-carrier) | | | | | |
| Alpha2B-adrenergic receptor (deletion | Insertion/deletion | 0.951 | 0.674 | 0.158 | 2.6 |
| carrier vs. non-carrier) | | | | | |
| Apolipoprotein B (thr carrier vs. non- | Thr98Ile (Thr71Ile) | 4.125 | 1.923 | 0.032 | 62 |
| carrier) | | | | | |
| DDAH1 (T homozygote vs. other) | IVS2-33C > T | 3.224 | 1.358 | 0.018 | 25 |
| Hypercholesterolemia in the family | NA | 1.130 | 0.627 | 0.072 | 3.1 |
| Smoking status (yes vs. no) | NA | 2.381 | 0.837 | 0.004 | 10.8 |
| CHD in the family (yes vs. no) | NA | 1.566 | 0.639 | 0.014 | 4.8 |
| History of cardiovascular disease | NA | 0.790 | 0.637 | 0.215 | 2.2 |
| Obesity in the family | NA | 1.179 | 0.634 | 0.063 | 3.3 |
| Waist-to-hip circumference ratio (cm/cm) | NA | 19.288 | 7.947 | 0.015 | >100 |

Constant −29.696.

TABLE 2
A multivariate logistic model predicting the risk of stroke.

| | | Coefficient | | | |
|---|---|---|---|---|---|
| Predictor | Mutation | (bᵢ) | S.E. | p-value | Odds ratio |
| Fibrinogen-beta (FGB) (G homozygote vs. | −455G > A | 3.838 | 1.626 | 0.018 | 46.4 |
| other) | | | | | |
| Alpha2B-adrenergic receptor (deletion | Insertion/deletion | 2.975 | 1.258 | 0.018 | 19.6 |
| homozygote vs. other) | | | | | |
| Neuropeptide Y (NPY) (C carrier vs. non- | −52C > G | 4.793 | 1.911 | 0.012 | 125 |
| carrier) | | | | | |
| Antihypertensive medication (yes vs. no) | NA | 2.282 | 1.063 | 0.032 | 9.8 |
| Smoking status (yes vs. no) | NA | 1.727 | 1.148 | 0.132 | 5.6 |
| Frequency of hangovers (per times/year) | NA | 0.114 | 0.093 | 0.217 | 1.12 |
| Body mass index (per kg/m²) | NA | 0.150 | 0.112 | 0.179 | 1.16 |

Constant −5.205.

### REFERENCES

Diverse Populations Collaborative Group. Prediction of mortality from coronary heart disease among diverse populations: is there a common predictive function? Heart 2002; 88: 222-8.
Liao Y, McGee D L, Cooper R S, Sutkowski M B. How generalizable are coronary risk prediction models? Comparison of Framingham and two national cohorts. Am Heart J 1999; 137: 837-45.
Nedelcheva Kristensen V, Kelefiotis D, Kristensen T and Borresen-Dale: High-Throughput methods for detection of genetic variation. Biotechniques 30:318-332, 2001.
Rabindranath K S, Anderson N R, Gama R, Holland M R. Comparative evaluation of the new Sheffield table and the modified joint British societies coronary risk prediction chart against a laboratory based risk score calculation. Postgrad Med J 2002; 78: 269-72.
Salonen J T. Is there a continuing need for longitudinal epidemiologic research—The Kuopio Ischaemic Heart Disease Risk Factor Study. Ann Clin Res 1988: 20: 46-50.
Salonen J T, Malin R, Tuomainen T-P, Nyyssonen K, Nissinen T, Lakka T A, Lehtimaki T. Polymorphism in the high density lipoprotein paraoxonase gene and the risk of acute myocardial infarction in men: a prospective population-based study. Brit Med J 1999; 319: 487-9.
Snapir A, Heinonen P, Tuomainen T-P, Alhopuro P, Karvonen M K, Lakka T A, Nyyssönen K, Salonen R, Kauhanen J, Valkonen V-P, Pesonen U, Koulu M, Scheinin M, Salonen J T. An Insertion/Deletion polymorphism in the α_{2B}-adrenergic receptor gene is a novel genetic risk factor for acute coronary events. J Am Coll Cardiol 2001; 37: 1516-1522.
Syvänen A-C: Accessing genetic variation: genotyping single nucleotide polymorphisms. Nature reviews/Genetics 2:930-942, 2001
Tuomainen T-P, Kontula K, Nyyssönen K, Lakka T A, Helio T, Salonen J T. Increased risk of acute myocardial infarction in carriers of the hemochromatosis gene Cys282Tyr mutation: A prospective cohort study in men in Eastern Finland. Circulation 1999; 100: 1274-1279.
Valkonen V-P, Päivä H, Salonen J T, Lakka T A, Lehtimäki T, Laakso J, Laaksonen R. Risk of acute coronary events in relation to concentration of serum asymmetrical dimethylarginine. Lancet 2001; 358: 2127-2128.

1. A method for diagnosing a susceptibility to cardiovascular disease especially myocardial infarction (MI) and stroke in a subject by detecting genetic variation or polymorphism, i.e. a mutation, in at least three of the genes selected from the group consisting of:
(a) α_{2B}-adrenoceptor
(b) apolipoprotein B
(c) dimethylarginine dimethylaminohydrolase 1
(d) fibrinogen-beta
(e) neuropeptide Y
(f) natriuretic peptide precursor A
(g) cystathione beta synthase
(h) glycoprotein IIb/IIIa
(i) lipoprotein lipase
comprising the steps of:
i) providing a biological sample of the subject to be tested,
ii) detecting the presence of mutations in the genes, the presence of a mutation in one or several of the genes indicating an increased risk of coronary heart disease (CHD) and/or myocardial infarction (MI) in said subject.

2. The method according to claim 1, wherein the detection step is a nucleic acid assay.

3. The method according to claim 2, wherein the detection step is carried out using a gene or DNA chip, microarray, strip, panel or similar combination of more than one genes, mutations or RNA expressions to be assayed.

4. The method according to claim 2, wherein the polymorphisms are determined using polymerase chain reaction.

5. The method according to claim 1, wherein the biological sample is a blood sample or buccal swab sample.

6. The method according to claim 1, further comprising a step of combining information concerning age, gender, the family history of cardiovascular diseases and hypercholesterolemia, and the medical history concerning cardiovascular diseases of the subject with the results obtained from step ii) of the method for confirming the indication obtained from the detection step.

7. The method according to claim 1, wherein said information is about hypercholesterolemia in the family, smoking status, CHD in the family, history of cardiovascular disease, obesity in the family, and waist-to-hip circumference ratio (cm/cm)

8. The method according to claim 1, wherein said information is about antihypertensive medication, smoking status, frequency of hangovers and body mass index.

9. The method according to claim 1, further comprising a step determining blood, serum or plasma cholesterol, HDL cholesterol, LDL cholesterol, triglyceride, apolipoprotein B and AI, fibrinogen, ferritin, transferring receptor, C-reactive protein, serum concentration or plasma insulin concentration.

10. The method according to claim 1, wherein the selected genes are natriuretic peptide precursor A, α_{2B}-adrenoceptor, apolipoprotein B and dimethylarginine dimethylaminohydrolase 1.

11. The method according to claim 1, wherein the selected genes are fibrinogen-beta, α_{2B}-adrenoceptor and neuropeptide Y.

12. The method according to claim 1 further comprising a step of determining height, weight, systolic and diastolic blood pressure, heart rate, maximal oxygen uptake, or other electrocardiographic measurement of the subject.

13. The method according to claim 10, wherein the detected mutations are Val32Met of natriuretic peptide precursor A, an insertion/deletion of three glutamic acids in the region of 12 Glu aminoacids in the codons 298-309 of α_{2B}-adrenoceptor, Thr98Ile of apolipoprotein B and SNP IVS2-33C>T of dimethylarginine dimethylaminohydrolase 1.

14. The method according to claim 11, wherein the detected mutations are SNP-455G>A of fibrinogen-beta, an insertion or deletion of three glutamic acids in the region of 12 Glu aminoacids in the codons 298-309 of α_{2B}-adrenoceptor, and SNP-52C>G of neuropeptide Y.

15. The method according to claim 1 further comprising a step of calculating the probability of a cardiovascular disease using a logistic regression equation as follows:
Probability of a cardiovascular disease=[1+e^{(-(-a+Σ(bi*Xi))}]⁻¹, where e is Napier's constant, Xᵢ are variables related to the cardiovascular disease, bᵢ are coefficients of these variables in the logistic function, and a is the constant term in the logistic function.

16. The method according to claim 15, wherein a and bᵢ are determined in the population in which the method is to be used.

17. The method according to claim 15, wherein Xi are selected among the variables that have been measured in the population in which the method is to be used.

18. The method according to claim 15, wherein bᵢ are between the values of −20 and 20.

19. The method according to claim 15, wherein Xᵢ are binary variables that can have values or are coded as 0 (zero) or 1 (one).

20. The method according to claim 15, wherein i are between the values 0 (none) and 100,000.

21. A kit for diagnosing a susceptibility to a cardiovascular disease especially myocardial infarction (MI) and stroke in a subject, comprising means for detecting genetic variation or polymorphism, i.e. a mutation, in at least three of the genes selected from the group consisting of:
(a) α_{2B}-adrenoceptor
(b) apolipoprotein B
(c) dimethylarginine dimethylaminohydrolase 1
(d) fibrinogen-beta
(e) neuropeptide Y
(f) natriuretic peptide precursor A
(g) cystathione beta synthase
(h) glycoprotein IIb/IIIa
(i) lipoprotein lipase
and optionally software to interpret the results of the detection.

22. The kit according to claim 21, comprising a DNA chip, microarray, DNA strip, DNA panel or real-time PCR based tests.

23. The kit according to claim 21, comprising a questionnaire for obtaining patient information concerning age, gender, height, weight, the family history of cardiovascular diseases and hypercholesterolemia, the medical history concerning cardiovascular diseases.
